(19) United States
(12) Patent Application Publication
(10) Pub. No.: US 2006/0039870 A1
(43) Pub. Date: Feb. 23, 2006

(54) Pulmonary delivery of enzymatic medical countermeasures

(76) Inventor: Jeffrey Donald Turner, Chute-A-Blondeau (CA)

Correspondence Address: Raymond J. Lillie, Esq.;c/o Carella, Byrne, Bain, Gilfillan,, Cecchi, Stewart & Olstein, 5 Becker Farm Road, Roseland, NJ, 07068, US

(21) Appl. No.: 11/195,041

(22) Filed: Aug. 2, 2005

Related U.S. Application Data
(60) Provisional application No. 60/603,186, filed on Aug. 20, 2004.

(51) Int. Cl. A61K 9/14; A61K 38/46; A61L 9/04

(52) U.S. Cl. 424/46; 424/94.6

(57) ABSTRACT

The present invention provides for non-invasive treatment of nerve agent poisoning by administering nerve agent neutralizing enzymes to the pulmonary epithelium of a subject, by inhalation, where they accumulate within the lungs. Localization of such enzymes in the pulmonary epithelium results in neutralization of the nerve agents at the lungs. As a result, nerve agents move by diffusion out of the blood through the pulmonary capillaries, duc to the organophosphorus nerve agents rapid diffusion across the cell membranes of the body via diffusion, down their concentration gradients. The present invention presents a practical method of administering nerve agent neutralizing enzymes, without requiring passage into the blood plasma, and without requiring blood plasma activity of the enzyme.

This application claims priority based on Provisional Application Ser. No. 60/603,186, filed Aug. 20, 2004, the contents of which are incorporated by reference in their entirety.

### FIELD OF THE INVENTION

This invention relates to a method of treating nerve agent poisoning comprising administration of a nerve agent neutralizing enzyme to the pulmonary system of a mammal by inhalation.

### BACKGROUND OF THE INVENTION

The use of organophosphate compounds in war and as pesticides has resulted in a rising number of cases of acute and delayed intoxication over the past 40 years, resulting in damage to the peripheral and central nervous systems, myopathy, psychosis, general paralysis, and death. It is estimated that 19,000 deaths occur out of the 500,000 to 1 million annual pesticide-related poisonings. In addition to these overt symptoms, animal studies have shown that administration of the organophosphatc methyl parathion suppressed growth and induced ossification in both mice and rats, and may cause malformations and fetal death in humans.

These cholinesterase-inhibiting substances prevent the breakdown of acetylcholine, resulting in a buildup which leads to hyperactivity of the nervous system. Acetylcholine is not destroyed and continues to stimulate the muscarinic receptor sites (exocrine glands and smooth muscles) and the nicotinic receptor sites (skeletal muscles).

Exposure to cholinesterase-inhibiting substances can cause symptoms ranging from mild (twitching, trembling) to severe (paralyzed breathing, convulsions), and in extreme cases, death, depending on the type and amount of cholinesterase-inhibiting substances involved. The action of cholinesteraseinhibiting substances such as organophosphates and carbamates makes them very effective as pesticides for controlling insects and other pests. Unfortunately, when humans breathe or are otherwise exposed to these compounds, they are subjected to the same negative effects. Mild poisoning occurs when blood cholinesterase activity is 20-50% of normal; moderate poisoning occurs when blood cholinesterase activity is 10-20% of normal; severe poisoning is characterized by blood cholinesterase activity of less than 10% of normal. Severe neuromuscular effects are observed when blood cholinesterase activity levels drop below 20% of normal, while levels near zero are generally fatal.

Indeed, the devastating impact of certain cholinesterase-inhibiting substances on humans has led to the development of these compounds as “nerve gases” or chemical warfare agents. Nerve agents are the most toxic chemical warfare agents. These compounds are related to organophosphorus insecticides, in that they are both esters of phosphoric acid. The major nerve agents are diisopropylfluorophosphate (DFP), GA (tabun), GB (sarin), GD (soman), CF (cyelosarin), GE, CV, yE, VG (amiton), VM, VR (RVX or Russian VX), VS, and VX. The nerve agents are classified into the C-series or V-series based upon their physical properties and toxieities. C-series nerve agents are volatile liquids at room temperature, and can be employed in liquid or vapor form. ½ series nerve agents, such as VX, are persistent liquid substances which can remain on material, equipment, and terrain for long periods. V-series nerve agents are generally more toxic than C-series nerve agents. Under temperate conditions, nerve agents are clear colorless liquids, which are difficult to detect.

Present treatment of organophosphate poisoning consists of post-exposure intravenous or intramuscular administration of various combinations of drugs, including carbamates (e.g., pyridostigmine), anti-muscarinics (e.g., atropine), and ChE-reactivators such pralidoxime chloride (2-PAM, Protopam). An anticonvulsive (e.g., diazepam) may also be administered. Although this drug regimen is effective in preventing death from organophosphate poisoning, it is not effective in preventing convulsions, performance deficits, or permanent brain damage. In addition, a post-exposure drug regimen is often useless because even a small dose of an organophosphate chemical warfare agent can cause irreversible acute poisoning before antidotes can be administered using conventional delivery systems.

These drawbacks have led to the investigation of eholinesterase enzymes for the treatment of organophosphate exposure. Post-exposure toxicology can be prevented by pretreatment with cholinesterases, which act to sequester the toxic organophosphates before they reach their physiological targets.

The use of cholinesterases as pre-treatment drugs has been successfully demonstrated in animals, including non-human primates. For example, pretreatment of rhesus monkeys with fetal bovine serum-derived AChE or horse serum-derived BChE protected them against a challenge of two to five times the LD5O of pinacolyl methylphosphonofluoridate (soman), a highly toxic organophophate compound used as a chemical weapon (Broomfield et al., J. Pharmaeol. Exp. Ther., 1991, 259:633-638; Wolfe et al., Toxicol, App]. Pharmaeol., 1992, 117(2):189-193). In addition to preventing lethality, the pretreatment prevented behavioral incapacitation after the soman challenge, as measured by the serial probe recognition task or the equilibrium platform performance task. Administration of sufficient exogenous human BChE can protect mice, rats, and monkeys from multiple lethal-dose organophosphate intoxication (See, e.g., Raveh et al,. Biochemical Pharmacology, 1993, 42:2465-2474; Raveh et al., Toxicol. Appl. Pharmacol., 1997, 145:43-53; Allon et al,, Toxicol. Sei., 1998, 43:121-128). Purified human BChE has been used to treat organophosphate poisoning in humans, with no significant adverse immunological or psychological effects (Cascio et al., Minerva Anestesiol., 1998, 54:337).

Titration of organophosphates both in vitro and in vivo demonstrates a 1:1 stoichiometry between organophosphate-inhibited enzymes and the cumulative dose of the toxic nerve agent. The inhibition of ChE by an organophosphate agent is due to the formation of a stable stoichiometric (1:1) covalent conjugate of the organophosphate with the ChE active site serine. Covalent conjugation is followed by a parallel competing reaction, termed “aging,” wherein the inhibited ChE is transformed into a form that cannot be regenerated by the commonly used reactivators. These reaetivators, such as active-site directed nucleophiles (e.g., quaternary oximes), normally detach the phosphoryl moiety from the hydroxyl group of the active site serine. The aging process us believed to involve dealkylation of the eovalently bound organophosphate group, and renders therapy of intoxication by certain organophosphates such as sarin, soman, and DFP exceedingly difficult.

Other enzymes have also demonstrated efficacy in neutralizing nerve agents. These enzymes include aryldialkylphosphatases (EC 3.1.8.1), organophosphate hydrolases (OPH), carboxylesterases (EC 3.1.1.1), triesterases, phosphotriesterases, arylesterases (EC 3.1.1.2), paraoxonases, diisopropylfluorophosphatases (DFPases, EC 3.1.8.2), and organophosphate acid anhydrases (OPAH). Certain forms of earboxylesterases have been shown to hydrolyze nerve agents such as sarin and soman, and have also been shown to confer immunity to pesticides (R. D. Newcomb et al., Proc. Natl. Acad. Sei. USA, 1997, 94:7464-7468). Paraoxonases have demonstrated a role in organophosphate metabolism, and grant resistance to organophosphate poisoning (J. E. Hulla et al., Toxicological Sciences, 1999, 47:135-143; L. C. Costa et al., Biomarkers, 2003, 8:1-12; C. Hassett et al., Biochemistry, 1991, 30: 10141-10149; S. Akgur et al., Forensic Sei. mt., 2003, 133(1-2):136-140; U.S. Pat. No. 5,629,193). Similarly, triesterases and phosphotriesterases have been shown to hydrolyze organophosphorus compounds (M. Sogorb et al., Toxicol. Lett., 2004, 151(1):219-233; D. Dumas, J. Biol. Chem., 1989, 264(33):19659-19665). Unlike eholinesterases, these enzymes do not demonstrate a 1:1 stoichiometry, and are not “aged” or inactivated by organophosphate compounds, but rather behave enzymatically.

Poisoning with organophosphate agents is a severe problem facing military personnel who may encounter lethal doses of these compounds in chemical warfare situations, While intravenous and intramuscular administration of BChE have been shown to be effective, they are not a practical method of drug delivery on the battlefield. The increasing need for alternate delivery systems for counteracting nerve agents is demonstrated by a small business innovation research (SBIR) solicitation by the United States Army (Department of Defense 2002.2 SBIR Solicitation A02-182, May 2, 2002, Developing Human-Compatible Needleless Delivery Systems for Administering Bioscavengers). The solicitation seeks alternatives to needle-based delivery systems for protein-based agents, in particular, for large molecular weight proteins. Specifically, the solicitation seeks a BChE formulation capable of delivering a systemic dose via the lungs, e.g., about 200 mg of an enzyme such as BChE in 4-5 inhalations. The solicitation also seeks to measure the potential efficacy of pulmonary delivery systems for delivery of enzymes into circulation. Thus, this proposal focuses on blood plasma activity of the administered enzyme.

A major hurdle for pulmonary delivery of large proteins is their poor absorption through the pulmonary epithelium. The nerve-agent neutralizing enzymes discussed above are large, oligomeric protein molecules that do not traverse the skin, gut, or pulmonary epithelia due to their large size and lipid insolubility. Similarly, enzymes in the blood will not cross the pulmonary epithelium into the lungs, which is the primary site of absorption of vapor nerve agents. Due to these limitations, administration of enzyme therapies to nerve agents under present understanding requires intravenous or intramuscular injection. Other technologies, such as patch technology, are presently infeasible, as the low diffusion coefficient across skin and other membranes requires an impracticably large dose of enzymes in the patch.

Thus, there is a continuing need for an efficient method of non-invasively administering enzymes that can neutralize nerve agents.

### SUMMARY OF THE INVENTION

As discussed above, present therapies for nerve agent poisoning require administration of nerve agent neutralizing enzymes into the bloodstream, which can be problematic and impractical in some conditions. The present invention involves the recognition that pulmonary accumulation of therapeutic enzymes for treating nerve agents is not a problem, but rather a solution; the present invention results from recognition, explained in detail below, that there is no need to deliver the enzymes systemically because they will act effectively on the pulmonary epithelia. The present invention provides for non-invasive treatment of nerve agents by administering nerve agent neutralizing enzymes by inhalation, where they accumulate within the lungs. The present invention presents a practical method of administering nerve agent neutralizing enzymes, without requiring passage into the blood plasma, and without requiring blood plasma activity of the enzyme.

Accordingly, the present invention is directed to a method of treating nerve agent poisoning in a subject comprising providing an effective amount of a nerve agent neutralizing enzyme, and delivering the nerve agent neutralizing enzyme to the pulmonary epithelium, e.g., by inhalation. Preferred subjects are humans, but other mammals, and indeed any animals with a developed lung that provides for extensive blood contact, can be treated by the invention.

In a preferred embodiment, the nerve agent neutralizing enzyme is present in a particle of a size of about 0.01 μm to about 4 μm, preferably from about 0.5 μm to about 1 μm. In a more preferred embodiment, the nerve agent neutralizing enzyme particle is about 1 μm.

In a specific embodiment, the nerve agent neutralizing enzyme is an aerosol form. In a further embodiment, the nerve agent neutralizing enzyme further comprises an excipient. In another specific embodiment, the nerve agent neutralizing enzyme is administered with an inhaler, in particular a metered dose inhaler.

Alternatively, the nerve agent neutralizing enzyme is in a liquid form. In a such an embodiment, the nerve agent neutralizing enzyme may further comprise an excipient. In a further embodiment of this aspect of the invention, the nerve agent neutralizing enzyme is administered with an inhaler or a nebulizer.

In still another embodiment, the nerve agent neutralizing enzyme is in a dry powder form. In such an embodiment, the nerve agent neutralizing enzyme may further comprise an excipient. In a further embodiment, the nerve agent neutralizing enzyme is administered with an inhaler.

In other embodiments, the nerve agent neutralizing enzyme is selected from the group consisting of cholinesterases, aryldialkylphosphatases, organophosphate hydrolases (OPH), carboxylesterases, triesterases, phosphotriesterases, arylesterases, paraoxonases, diisopropylfluorophosphatases, and organophosphate acid anhydrases. Preferably, the organophosphate hydrolases (OPH), carboxylesterases, triesterases, phosphotriesterases, paraoxonases, diisopropylfluorophosphatases, or organophosphate acid anhydrases may be delivered in doses of from about 0.1 mg to about 30 mg, and more preferably, from about 1 mg to about 5 mg.

In a specific embodiment, the nerve agent neutralizing enzyme is a cholinesterase. For example, as exemplified below, the nerve agent neutralizing enzyme can be butyrylcholinesterase. The cholinesterase may be delivered in doses of from about 1 mg to about 250 mg, and more preferably from about 25 mg to about 150 mg.

in another embodiment, the present invention is directed to a pharmaceutical dosage form for delivery of a nerve neutralizing enzyme to the pulmonary system of a mammal, the form comprising a therapeutically effective amount of the enzyme and a pharmaceutically acceptable carrier or diluent, wherein said dosage form is contained in an inhaler or nebulizer.

### DETAILED DESCRIPTION OF THE INVENTION

It has been discovered that administration by inhalation of nerve agent neutralizing enzymes and localization of such enzymes in the pulmonary epithelium results in neutralization of the nerve agents by diffusion out of the blood through the pulmonary capillaries. Organophosphorus nerve agents have been specifically designed to diffuse rapidly across the cell membranes of the body via diffusion, down their concentration gradients. Nerve agent neutralizing enzymes dissolved in the alveolar fluid on the lumen surface of the lung's epithelial cells, by virtue of their ability to react with the nerve agents, either stoieheometrically or catalytically, keep the level of nerve agents in the lumenal side of the respiratory membrane at a very low level. This creates a diffusion gradient for nerve agents out of the blood. As a result, the nerve agents move out of the blood and are neutralized at the pulmonary epithelium without requiring the nerve agent neutralizing enzymes to enter the bloodstream.

Nerve agent neutralizing enzymes present in the alveolar fluid on the lumen surface of the lung's epithelial cells also create a chemical ban-ier, inactivating inhaled nerve agents at the respiratory membrane before they are absorbed into the blood. Thus, in the case of inhaled nerve agents, no build-up of nerve agent would be created and thus no significant amounts of nerve agent would cross into the blood and cause toxic effects.

Thus, according to the invention, and contrary to present understanding, it is not necessary for the nerve agent neutralizing enzymes to gain access to the circulation to be functional. The present invention relies upon the nerve agent's hi-directional freedom of movement, and the nerve agent neutralizing enzymes remaining exclusively in the apical surface of the lung due to their size. The relatively large molecular weight and lipophobic properties of the nerve agent neutralizing enzymes prevent them from diffusing across the respiratory membrane and into the blood.

In short, the nerve agent neutralizing enzymes do not need to gain access into the blood to exert their protective effect, and therefore need not move across the pulmonary membranes; it is the nerve agents that move freely across the membranes due to their lipophilie properties.

The concept of pulmonary bi-directional movement is well known in the art. For example, during surgery, anesthesia such as halothane is inhaled into the lungs. The anesthetic crosses the respiratory membrane quickly, moving down its concentration gradient and into the blood and brain. When the surgery is completed, anesthesia administration is halted, which results in the partial pressure of halothane falling in the lungs as they are ventilated. This creates a concentration gradient from blood to lung lumen, which causes diffusion of the anesthesia out of the blood and into the lungs, where they are exhaled. More than 80% of the clearance of inhaled anesthetics, like halothane, is via expired air. The halothane molecules leave the central nervous via diffusion, enter the blood and then are lost at the lungs. (Goodman and Gilman, The Pharmacological Basis of Therapeutics, 1980, MacMillan Publishing Co, New York).

The magnitude and direction of the diffusion of molecules is expressed mathematically as:
M=C A(D1−D2)/H
wherein M is the mass of substance diffusing, C is the coefficient of diffusion, A is the area of diffusion surface, D1 is the concentration of substance on one side of the membrane, D2 is the concentration of substance on the opposite side of D1, and H is the thickness of the diffusion membrane.

This equation applies to removing nerve agent from the body via the lungs in the following manner. To maximize M, it is desirable to have a large surface area for diffusion (A), a large concentration gradient (D1−D2) of a substance that diffuses easily (C), and the thinnest membrane (H) possible. The lungs have a very large surface area with an extremely thin respiratory membrane. Nerve agents were specifically designed to have a high diffusion coefficient (C) and traverse the respiratory membrane quickly. Thus, the presence of nerve agent neutralizing enzymes on the alveolar lumen will ensure that the concentration of nerve agent (D2) approaches zero, thus always drawing the nerve agent out of the blood (D1), across the respiratory membrane easily and into the lung lumen, where the nerve agent is destroyed by accumulated nerve agent neutralizing enzymes.

The lungs are an ideal site for nerve agent enzymatic inactivation for a number of reasons. The lungs provide an extensive surface area, i.e., the human respiratory membrane averages 70 square meters. The respiratory membrane is very thin, designed for high molecular diffusion, and can be as thin as 0.2 μm, with a mean thickness of 0.5 μm. Pulmonary lumen is not in direct contact with the blood, so enzymes located on the lung's epithelial cells are less likely to be inactivated by antibodies upon repeated dosing. The lungs are also well vascularized and well perfused, resulting in contact with a high volume of blood plasma. Thus, nerve agent neutralizing enzymes accumulated on the lung epithelium will come into contact with a large volume of nerve agents from blood plasma. During stress, cardiac output rises and further increases perfusion of the lungs, thus speeding the inactivation of nerve agents.

The method of the present invention provides numerous advantages over present nerve agent poisoning therapies. The present invention avoids the need to intravenously or intramuscularly inject nerve agent neutralizing enzymes, which can be advantageous in situations where the use of needles and syringes is impractical, such as when subjects are wearing chemical protection suits. Pulmonary administration also allows self-administration of multiple doses, and eliminates the need for specialized techniques or the presence of medical personnel, who may not be available, e.g., on a battlefield. The individual dosage size may also be customized, allowing dosing to effect on an individual basis, and supplemental dosing in cases of worsening toxicity. Modem pulmonary delivery devices, such as inhalers and nebulizers, are portable and convenient, and can deliver up to 30 mg of drug per inhalation, although as a practical matter lower amounts, e.g., 5 mg, are more usual. Indeed, the ability to deliver multiple doses to the lung overcomes practicalities and limitations of current pulmonary delivery technology. Further, intubation and mechanical ventilation can be used to introduce the nerve agent neutralizing enzyme to infants, small children, and patients suffering severe illness or incapacitation.

An additional advantage of the method of the present invention is the speed of efficacy of the treatment. All cutaneous nerve agents enter the body either across the skin, through the pulmonary system, or through the gastrointestinal tract. Once in the bloodstream, the nerve agent travels first to the lungs via the pulmonary circulation, where the nerve agents can be destroyed by the accumulated nerve agent neutralizing enzymes, before the blood is then distributed systemically to the body. As noted above, pulmonary administration also allows for direct neutralization of high vapor pressure nerve agents, such as sarin, which enter the body principally via the pulmonary route.

An additional advantage of the method of the present invention is the extended half-life of the nerve agent neutralizing enzymes. As noted above, pulmonary administration to the apical surface avoids contact with lung leukocytes, and thus may limit inmmnogenicity due to the limited action of maerophages in the lung. This limits immunologic inactivation of the nerve agent neutralizing enzymes during multiple administrations, and may also allow the use of non-human forms of the nerve agent neutralizing enzymes.

Nerve agent neutralizing enzymes in the form of lyophilized powders have long stability, a critical point for forward deployment or first responder communities where stockpiled medicines may be forward deployed but not be used for years. Also, since the nerve agent neutralizing enzymes need not enter the bloodstream, the half-life of the nerve agent neutralizing enzymes in the blood is less important.

### Definitions

The following defined terms are used throughout the present specification, and should be helpful in understanding the scope and practice of the present invention.

By “inhalation” or “pulmonary administration” is meant administration to the lung epithelium, e.g., by use of an inhaler or nebulizer. The formulation may utilize aerosolized particles or may include an aerosolizing agent. Alternatively, the formulation may utilize a dry powder and optionally an excipient. Alternatively, the formulation may utilize a liquid and optionally an excipient.

By “aerosolized” is meant that a compound must be broken down to liquid or solid particles in order to ensure that the dose actually reaches the mucous membranes of the nasal passages or the lung. The term “aerosol particle’ is used herein to describe the liquid or solid particle suitable for nasal or pulmonary administration, i.e., that will reach the mucous membranes.

By “pulmonary delivery device” is meant a device for pulmonary administration, i.e., administration that will reach the mucous membranes of the lungs. By way of example, a pulmonary delivery device includes but is not limited to inhalers, such as metered dose inhalers or dry powder inhalers, and nebulizers. The pulmonary delivery device may use a propellant or aerosolizing agent.

Nerve agents” are substances, generally prepared by chemical synthesis or extraction from natural sources, that may cause deleterious or undesirable effects to a living creature if inhaled, absorbed, ingested, or otherwise encountered because of their high reactivity with and inhibition of cholinesterases, e.g., as discussed in the Background of the Invention. These agents include all of the agents discussed in the Background, e.g., organophosphorus compounds, such as diisopropylfluorophosphate (DFP), CA (tabun), GB (sam), GD (soman), GE (cyclosarin), GE, CV, yE, VG (amiton), VM, VR (RVX or Russian VX), VS, VX, and combinations thereof. The foregoing list is exemplary and not limiting.

By “nerve agent poisoning” is meant deleterious or undesirable effects to a living creature exposed to a nerve agent or an organophosphorate pesticide. Organophosphate pesticides include acephate, azinphos-methyl, bensulide, cadusafos, chlorethoxyfos, chlorpyrifos, chlorpyrifos methyl, chlorthiophos, coumaphos, dialiflor, diazinon, diehlorvos (DDVP), dierotophos, dimethoate, dioxathion, disulfoton, ethion, ethoprop, ethyl parathion, fenamiphos, fenitrothion, fenthion, fonofos, isazophos methyl, isofenphos, malathion, methamidophos, methidathion, methyl parathion, mevinphos, monocrotophos, naled, oxydemeton methyl, phorate, phosalone, phosmet, phosphamidon, phostebupirim, pirimiphos methyl, profenofos, propetamphos, sulfotepp, sulprofos, temephos, terbufos, tetraehlorvinphos, tribufos (JDEF), trichlorfon. The foregoing list is exemplary and not limiting.

By “nerve agent neutralizing enzyme” is meant a protein or polypeptide capable of reducing the toxicity of a nerve agent or organophosphate pesticide. Alternatively, a nerve agent neutralizing enzyme may be a protein or polypeptide which can cause an improvement in a clinically significant condition in the host caused by exposure to nerve agents. These enzymes include, but are not limited to, all of the enzymes discussed in the Background, e.g., cholinesterases, aryldialkylphosphatases, organophosphate hydrolases (OPH), carboxylesterases, triesterases, phosphotriesterases, arylesterases, paraoxonases, diisopropylfluorophosphatases, organophosphate acid anhydrase, and combinations thereof.

The phrase “therapeutically effective amount” is used herein to mean an amount sufficient to cause an improvement in a clinically significant condition in the host. For example, a therapeutically effective amount can be an amount sufficient to reduce by about 15 percent, preferably by about 50 percent, more preferably by about 90 percent. and most preferably prevent, a clinically significant deficit in the activity, function and response of the host.

The term “particle size” roughly means the diameter of the particle, or the longest axial distance of the particle if the particle is not spherical. It should be understood that on a microscopic scale dry powder particles will have irregular shape.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness, and the like, when administered to a human. Preferably, as used herein, the term “pharmaceutically acceptable” means approved by a regulatory agency of the Federal or a state government or listed in the U.S. Pharmacopeia or other generally recognized phammcopeia for use in animals, and more particularly in humans. The term “carrier” refers to a diluent, adjuvant, excipient, or vehicle with which the compound is administered. Such pharmaceutical caters can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “subject” as used herein refers to a mammal (e.g., rodent such as a mouse or rat, pig, primate, companion animal (e.g., dog or cat, etc.). In particular, the term refers to a human.

The terms “about” and “approximately” mean within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to ±5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Nerve Agent Neutralizing Enzymes

The present invention encompasses multiple enzymes capable of neutralizing or degrading nerve agents. These agents include all of the enzymes discussed in the background, e.g., cholinesterases, aryldialkylphosphatases, organophosphate hydrolases (OPH), carboxylesterases, triesterases, phosphotriesterases, arylesterases, paraoxonases, diisopropylfluorophosphatases, and organophosphate acid anhydrases. In one embodiment, the present invention provides for the use of a cholinesterase. In another embodiment, the present invention provides for the use of butyrylcholinesterase. These nerve agent neutralizing enzymes may operate in a stoichiometric ratio, by binding and inactivating nerve agents in a 1:1 ratio. These nerve agent neutralizing enzymes may also operate by enzymatically cleaving nerve agents, and may inactivate nerve agents in a ratio of one nerve agent neutralizing enzyme to twenty or more nerve agent molecules. The present invention also provides for a nerve agent neutralizing enzyme administered via an inhaler or nebulizer.

(a) Cholinesterases

The general term cholinesterase (ChE) refers to a family of enzymes involved in nerve impulse transmission. The major function of ChE enzymes is to catalyze the hydrolysis of the chemical compound acetylcholine at the cholinergic synapses. Electrical switching centers, called synapses, are found throughout the nervous systems of humans, other vertebrates and insects. Muscles, glands, and neurons are stimulated or inhibited by the constant firing of signals across these synapses. Stimulating signals are carried by the neurotransmitter acetylcholine, and discontinued by the action of ChE enzymes, which cause hydrolytic breakdown of acetylcholine. These chemical reactions occur continuously at a very fast rate, with acetylcholine causing stimulation and ChE enzymes ending the signals. The action of ChE allows the muscle, gland, or nerve to return to its resting state, ready to receive another nerve impulse if need be.

Cholinesterases are classified into two broad groups, depending on their substrate preference and sensitivity to selective inhibitors. Those enzymes which preferentially hydrolyze acetyl esters such as acetylcholine, and whose enzymatic activity is sensitive to the chemical inhibitor BW 284C5 1, are called acetylcholincsterases (AChE), or acetylcholine acetylhydrolase (BC 3.1.1.7). Those enzymes which preferentially hydrolyze other types of esters such as butyrylcholine, and whose enzymatic activity is sensitive to the chemical inhibitor tetraisopropylpyrophosphoramide (also known as iso-OMPA), are called butyrylcholinesterases (BChE, BC 3.1.1.8). BChE is also knowD as pseudocholinesterase or non-specific cholinesterase. Further classifications of ChE's are based on charge, hydrophobicity, interaction with membrane or extracellular structures, and subunit composition.

Acetylcholinesterase (AChE), also known as true, specific, genuine, erythrocyte, red cell, or Type I ChE, is a membrane-bound glycoprotein and exists in several molecular forms. It is found in erythrocytes, nerve endings, lungs, spleen, and the gray matter of the brain. Butyrylcholinesterase (BChE), also known as plasma, serum, benzoyl, false, or Type II ChE, has more than eleven isoenzyme variants and preferentially uses butyrylcholine and benzoylcholine as in vitro substrates. BChE is found in mammalian blood plasma, liver, pancreas, intestinal mucosa, the white matter of the central nervous system, smooth muscle, and heart. BChE is sometimes referred to as serum cholinesterase as opposed to red cell cholinesterase (AChE).

AChE and BChE exist in parallel arrays of multiple molecular forms composed of different numbers of catalytic and non-catalytic subunits. Both enzymes are composed of subunits of about 600 amino acids each, and both are glycosylated. ACHE may be distinguished from the closely related BChE by its high specificity for the acetylcholine substrate and sensitivity to selective inhibitors. While ACHE is primarily used in the body to hydrolyze acetylcholine, the specific function of BChIE is not as clear. BChE has no known specific natural substrate, although it also hydrolyzes acetylcholine.

By “butyrylcholinesterase enzyme” or “BChE enzyme” is meant a polypeptide capable of hydrolyzing acetylcholine and butyryleholine, and whose catalytic activity is inhibited by the chemical inhibitor iso-OMPA. Preferred BChE enzymes to be produced by the present invention are mammalian BChE enzymes. Preferred mammalian BChE enzymes include human BChE enzymes. The term “BChE enzyme” also encompasses pharmaceutically acceptable salts of such a polypeptide.

By “recombinant butyryleholinesterase” or “recombinant BChE” is meant a BChE enzyme produced by a transiently transfected, stably transfected, or transgenic host cell or animal. The term “recombinant BChE” also encompasses pharmaceutically acceptable salts of such a polypeptide. Recombinant butyrylcholinesterase is well known in the art and is readily available (Arpagns et al, Biochemistry, 1990, 29:124-13 1; U.S. Pat. No. 5,215,909; Soreq et al., J. Biol. Chem., 1989, 264:10608-10613; Soreq et al., EMBO Journal, 1984, 3(6)1371-1375). In a preferred embodiment, recombinant BChE is obtained in high yield from the milk or urine of transgenic animals (PCT Publication No. WO 03/054182).

Butyrylcholinesterase derived from human serum is a globular, tetrameric molecule with a molecular mass of approximately 340 kDa. Nine Asn-linked carbohydrate chains are found on each 574-amino acid subunit. The tetrameric form of BCEE is the most stable and is preferred for therapeutic purposes. Wildtype, variant, and artificial BChE enzymes can be produced by one skilled in the art.

By “recombinant acetylcholinesterase” or “recombinant AChE” is meant an AChE enzyme produced by a transiently transfected, stably transfected, or transgenic host cell or animal. The term “recombinant AChE” also encompasses pharmaceutically acceptable salts of such a polypeptide. Recombinant acetylylcholinesterase is well known in the art and is readily available (European Pat. Nos. 114,756 and 388,906).

Preferably, the BChE or ACHE enzyme utilized according to the method of the present invention comprises an amino acid sequence that is substantially identical to a sequence found in a mammalian BChE or AChE, respectively. More preferably, the BChE or AChE sequence is substantially identical to the human BChE or ACHE, respectively. The BChE of the invention may be produced as a tetramer, a trimer, a dimer, or a monomer. In a preferred embodiment, the BCKE or AChE of the invention has a glycosylation profile that is substantially similar to that of native human BChE or AChE, respectively.

(b) Tetrameric BChE

The BChE utilized in the present invention may be in tetrameric form. It is believed that the tetrameric form of BChE is more stable and has a longer half-life in the plasma, thereby increasing its therapeutic effectiveness. BChE expressed recombinantly in CR0 (Chinese hamster ovary) cells was found not to be in the more stable tetrameric form, but rather consisted of approximately 55% dimers, 10-30% tetramers and 15˜40oo monomers (Blong et al., Biochem. J., 1997, 327:747-757). Recent studies have shown that a proline-rich amino acid sequence from the N-terminus of the collagen-tail protein caused aeetylcholinesterase to assemble into the tetrameric form (Bon et al., J. Biol. Chem., 1997, 272(5):3016-3021 and Krejci et al., J. Biol. Chem., 1997, 272:22840-22847). Thus, to increase the amount of tetrameric BChE enzyme, the DNA sequence encoding the BChE enzyme of the invention may comprise a proline-rich attachment domain (PRAD), which recruits recombinant BChE subunits (e.g., monomers, dimers and trimers) to form tetrameric associations.

(c) Non-Tetrameric BChE

Other forms of the BChE (e.g., monomers, dimers and trimers) have demonstrated substrate activity and are also encompassed by the invention. However, the observation that non-tetrameric forms of BChE are less stable in vivo does not rule out their usefulness in in vivo applications. Higher doses or more frequent in vivo administration of the non-tetrameric forms of BChE can result in satisfactory therapeutic activity.

(d) Other Nerve A2ent Neutralizin2 Enzymes

Carboxylesterases are enzymes closely related to cholinesterases which bind to organophosphorus and nerve agents. Carboxylesterases display stoichiometric activity similar to cholinesterases, and experience the same “aging” process when in contact with nerve agents. However, mutant forms of carboxylesterase display the ability to hydrolyze organophospates, resulting in resistance to the nerve agent in certain organisms. (R. D. Newcomb et al., Proc. Natl. Acad. Sci. USA, 1997, 94:7464-7468). Expression levels for carboxylesterases vary between different species, resulting in varying resistance to organophosphates between species. For examples, rodents express higher levels of carboxylesterases relative to humans, and accordingly are more resistant to organophosphate poisoning. As disclosed herein, one skilled in the art will be capable of administering carboxylesterases according to the method of the present invention.

Paraoxonases, also called arylesterases, are enzymes that hydrolyzes the toxic metabolites (oxons) of various organophosphate compounds and nerve agents. Paraoxonase is found predominantly in the liver and blood, and displays varying levels of activity between species. The enzymatic activity of paaoxonases protects from the neurotoxic effects of organophosphorus compounds, and can grant resistance to exposure to nerve agents (11. E. Hulla et al., Toxicological Sciences, 1999, 47:135-143; L. G. Costa et al., Biomarkers, 2003, 8:1-12; C. Hassett et al., Biochemistry, 1991, 30: 10141-10149; U.S. Pat. No. 5,629,193). Methods of generating recombinant paraoxonases and purifying paraoxonases are well known in the art, and paraoxonase is readily available. K. N. Gan et al., Drug. Metab. Dispos., 1991, 19(1):100-106; C. Hassett et al., Biochemistry, 1991, 30(42):10141-10149; U.S. Pat. No. 5,629,193). As disclosed herein, one skilled in the art will be capable of administering paraoxonases according to the method of the present invention.

Other enzymes, such as triesterases and phosphotriesterases, have been shown to have similar properties in hydrolyzing organophosphorus compounds (M. Sogorb et al., Toxicol. Lett., 2004, 151(1):219-233; D. Dumas et al., J. Biol. Chem., 1989, 264(33):19659-19665). As disclosed herein, one skilled in the art will be capable of administering triesterases, and phosphotriesterases according to the method of the present invention.

Another enzyme known to degrade nerve agents includes diisopropylfluorophosphatase (DFPase; EC 3.1.8.2), which is known to degrade sam, cyclosarin, soman, and VX. The DFPase gene has been isolated, and recombinant forms are well known in the art. (Hartleib et al., Protein Expression & Purification, 2001, 21:210-219; German patent DE19808192, to Ruterjans et al.).

Pulmonary Delivery Devices

Pulmonary delivery devices for administration of active agents are well known in the art. Pulmonary delivery devices generate particles of active agent, typically about 0.01 μm to about 4 μm, which may be inhaled by the subject. Pulmonary delivery devices are widely used for inhalation of an active agent from solution or suspension, or inhalation of an active agent in dry powder form, optionally admixed with an excipient. Examples of pulmonary delivery devices include, but are not limited to, metered dose inhalers (MDIs), nebulizers, and dry powder inhalers (DPJ5). The pulmonary delivery devices may optionally be pressurized, and may utilize propellant systems. The pulmonary delivery devices may also incorporate holding chambers, e.g., spacers, to prevent aerosolized active agents from escaping into the air, and allowing the subject more time to inhale.

Pulmonary delivery devices are well known in the art to provide numerous advantages over other delivery methods. Pulmonary delivery devices are well known to provide local effects in the lungs and pulmonary system by delivering active agents, including chemical compounds, antibodies, polypeptides, and proteins. Pulmonary delivery devices allow for higher bioavailability of an active agent due to the large surface area of the pulmonary epithelium, resulting in lower doses and fewer side effects. Furthermore, pulmonary delivery devices are cost-effective, easy to use, and are non-invasive.

Pulmonary delivery devices utilized in the method of the present invention may be activatable by inhalation, e.g., will automatically dispense active agent upon inhalation by the subject, and may be used with aerosol containers which contain active agents and optionally contain propellants. These devices can administer a plurality of metered doses in a controlled manner, allowing controlled and consistent dosing of active agents into the subject's bronchial passages and pulmonary epithelium. Examples of pulmonary delivery devices are described in U.S. Pat. Nos. 5,290,539, 6,615,826, 4,349,945, 6,460,537, 6,029,661, 5,672,581, 5,586,550, and 5,511,540, which are incorporated by reference herein.

MDIs operate by utilizing a propellant to eject a constant volume of an active agent, which is inhaled by the subject. MDIs may also include a surfactant to prevent aggregation of the active agent. The active agent may be dissolved or suspended in solution. MDIs utilizing propellants may require simultaneous inhalation and activation of the MDI. Holding chambers, e.g., spacers, may be used to store the aerosolized active agent, eliminating the need for simultaneous activation and inhalation. MDIs provide a constant, metered dosage of the active agent and allow for consistent dosing. Examples of MDIs are described in U.S. Pat. Nos. 6,615,826 and 5,290,539.

Nebulizers operate by creating a mist, i.e., nebulizing or atomizing, a formulation of active agent in solution, which is inhaled by the subject. The active agent may be dissolved or suspended in solution. The droplets may be created by any method known in the art, including the use of a fan, a vibrating member, or ultrasonic apparatus. Nebulizers are more gentle than MDIs and DPIs, and are appropriate for individuals unable to use inhalers, such as infants, young children, and individuals that are seriously ill or incapacitated. Examples of nebulizers are described in U.S. Pat, Nos. 6,748,945, 6,530,370, 6,598,602, and 6,009,869.

DPIs do not use propellants, and administer dry powder which is inhaled by the subject. To distribute the dry powder, DPIs may utilize any method known in the art to propel the active agent, including pneumatic systems, powered fans, or mechanical propulsion, e.g., squeezing of the container. DPIs may instead rely simply upon the inhalation by the subject. Blending of active agent with propellants is not required for DPIs, allowing delivery of larger payloads of active agent. An example of a DPI is described in U.S. Pat. No. 6,029,661.

Often, the aerosolization of a liquid or a dry powder formulation for inhalation into the lung will require a propellant. The propellant may be any propellant generally used in the art. Specific non-limiting examples of such useful propellants are a chlorotlourocarbon, a hydrofluorocarbon, a hydochlorofluorocarbon, or a hydrocarbon, including triflouromethane, dichlorodiflouromethane, dichlorotetrafiioroethanol, and 1,1,1,2-tetraflouroethane, or combinations thereof. Examples of propellant formulations are described in U.S. Pat, No. 5,672,581, which is incorporated herein by reference.

The present invention also encompasses other methods known in the art for pulmonary administration. By way of example, these methods include delivery by intratracheal inhalation, insufflation, or intubation, e.g., the delivery of a solution, a powder, or a mist into the lungs by a syringe, tube, or similar device.

Method of Administering Cholinesterases of the Present Invention

The present invention provides for a method of administration of nerve agent neutralizing enzymes to the pulmonary epithelium of a subject. The present invention encompasses any method known in the art for pulmonary delivery, including the pulmonary delivery devices and techniques described above. Nerve agent neutralizing enzymes of the present invention are administered by inhalation to the pulmonary epithelium. Inhalation may be oral or nasal. Nerve agent neutralizing enzymes may be administered via an MDI, nebulizer, or DPI, Multiple nerve agent neutralizing enzymes may be administered simultaneously. It is to be understood that more than one active agent may be incorporated in addition to the nerve agent neutralizing enzyme and that the use of the term “nerve agent neutralizing enzyme in no way excludes the use of additional active agents.

A nerve agent neutralizing enzyme as described herein can be present within a pharmaceutical composition. A pharmaceutical composition comprises a nerve agent neutralizing enzyme in combination with one or more pharmaceutically or physiologically acceptable carriers, diluents, or excipients. Such compositions may comprise buffers (e.g., neutral buffered saline or phosphate buffered saline), carbohydrates (e.g., glucose, mannose, sucrose or dextrose), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate.

Nerve agent neutralizing enzyme formulations suitable for use in the present invention include dry powders, solutions, suspensions or slurries for nebulization, and particles suspended or dissolved within a propellant. Dry powders suitable for use in the present invention include amorphous active agents, crystalline active agents and mixtures of both amorphous and crystalline active agents. The dry powder active agents have a particle size selected to prevent penetration into the alveoli of the lungs, that is, preferably from about 0.01 μm to about 4 μm, preferably less than 3 μm, more preferably from about 0.5 μm to about 1 μm, and most preferably about 1 μm in diameter. These dry powder active agents have a moisture content below about 10% by weight, usually below about 5% by weight, and preferably below about 3% by weight. Such active agent powders are described in WO 95/24183 and ‘NO 96/32149, which are incorporated by reference herein.

Dry powder nerve agent neutralizing enzyme formulations may be prepared by spray drying under conditions which result in a substantially amorphous powder. Bulk nerve agent neutralizing enzyme, which may be in crystalline form, is dissolved in a physiologically acceptable aqueous buffer, typically a citrate buffer having a pH range from about 2 to 9. The nerve agent neutralizing enzyme is dissolved at a concentration from 0.01% by weight to 1% by weight, usually from 0.1% to 0.2%. The solutions may then be spray dried in a conventional spray drier available from commercial suppliers such as Niro A/S (Denmark), Buchi (Switzerland) and the like, resulting in a substantially amorphous powder. These amorphous powders may also be prepared by lyophilization, vacuum drying, or evaporative drying of a suitable active agent solution under conditions to produce the amorphous structure. The amorphous nerve agent neutralizing enzyme formulation so produced can be ground or milled to produce particles within the desired size range. Dry powder nerve agent neutralizing enzymes may also be in a crystalline form. The crystalline dry powders may be prepared by grinding or jet milling the bulk crystalline active agent.

The nerve agent neutralizing enzyme powders of the present invention may optionally be combined with pharmaceutical eaters or excipients which are suitable for respiratory and pulmonary administration. Such carriers may serve simply as bulldng agents when it is desired to reduce the active agent concentration in the powder which is being delivered to a patient, but may also serve to improve the dispersability of the powder within a powder dispersion device in order to provide more efficient and reproducible delivery of the active agent and to improve handling characteristic of the nerve agent neutralizing enzyme such as floxyability and consistency to facilitate manufacturing and powder filling. Such excipients include but are not limited to (a) carbohydrates, e.g., monosaccharides such as fructose, galactose, glucose, D-mannose, sorbose, and the like; disaccharides, such as lactose, trehalose, cellobiose, and the like; cyclodextrins, such as 2-hydroxypropyl-.beta.-cyclodextrin; and polysaccharides, such as raffinose, maltodextrins, dextrans, and the like; (b) amino acids, such as glycine, arginine, aspartic acid, glutamic acid, cysteine, lysine, and the like; (c) organic salts prepared from organic acids and bases, such as sodium citrate, sodium ascorbate, magnesium gluconate, sodium gluconate, tromethamin hydrochloride, and the like; (d) peptides and proteins such as aspartame, human serum albumin, gelatin, and the like; and (e) alditols, such as mannitol, xylitol, and the like. A preferred group of caters includes lactose, trehalose, raffinose, maitodextrins, glycine, sodium citrate, human serum albumin and mannitol.

The amount of nerve agent neutralizing enzyme to be administered will be that amount necessary to deliver a therapeutically effective amount of the nerve agent neutralizing enzyme to achieve the desired result. In practice, this will vary widely depending upon the particular nerve agent neutralizing enzyme, the severity of the condition, the weight of the subject, and the desired therapeutic effect. In practice, the dose of nerve agent neutralizing enzyme may be delivered in one or more doses.

The nerve agent neutralizing enzyme compositions of the present invention may be in aerosol form. The liquid aerosol formulations contain the compounds of the present invention and a dispersing agent in a physiologically acceptable diluent. The dry powder aerosol formulations of the present invention consist of a finely divided solid form of the compounds of the present invention and a dispersing agent. With either the liquid or dry powder aerosol formulation, the formulation must be aerosolized. Other considerations, such as construction of the delivery device, additional components in the formulation, and particle characteristics are important. These aspects of nasal or pulmonary administration of a drug are well known in the art, and manipulation of formulations, aerosolization means and construction of a delivery device require at most routine experimentation by one of ordinary skill in the art.

The nerve agent neutralizing enzyme compositions of the present invention may be suspended, dispersed, or dissolved in solution. The liquid cater or intermediate can be a solvent or liquid dispersive medium that contains, for example, water, ethanol, a polyol (e.g. glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants. Prevention of the action of microorganisms can be achieved by the addition of various antibacterial and antifungal agents, e.g. paraben, chlorobutanol, or sorbic acid. In many cases isotonic substances are recommended, e.g. sugars, buffers and sodium chloride to assure osmotic pressure similar to those of body fluids, particularly blood.

Sterile solutions can also be prepared by mixing the nerve agent neutralizing enzyme formulations of the present invention with an appropriate solvent and one or more of the aforementioned excipients, followed by sterile filtering. In the case of sterile powders suitable for use in the preparation of sterile injectable solutions, preferable preparation methods include drying in vacuum and lyophilization, which provide powdery mixtures of the isostructural pseudopolymorphs and desired excipients for subsequent preparation of sterile solutions.

Appropriate dosages and the duration and frequency of administration will be determined by such factors as the condition of the patient, the type and severity of the patient's disease and the method of administration. In general, an appropriate dosage and treatment regimen provides the nerve agent neutralizing enzyme in an amount sufficient to provide therapeutic and/or prophylactic benefit. Various considerations for determining appropriate dosages are described, e.g., in Goodman and Gilman, The Pharmacological Basis of Therapeutics, 1980, MacMillan Publishing Co, New York. Appropriate dosages may generally be determined using experimental models and/or clinical trials. In general, the use of the minimum dosage that is sufficient to provide effective therapy is preferred. Patients can be monitored for therapeutic effectiveness using physical examination, imaging studies, or assays suitable for the condition being treated or prevented, which will be familiar to those of ordinary skill in the art. Dose adjustments can be made based on the monitoring findings. For example, an individual with exposure to nerve agent, following administration of nerve agent neutralizing enzyme according to the invention, for cessation of symptoms caused by the nerve agent. Based upon the foregoing considerations, determination of appropriate dosages will require no more than routine experimentation by those of ordinary skill in the art.

In a specific embodiment, the dosage is administered as needed. One of ordinary skill in the art can readily determine a volume or weight of nerve agent neutralizing enzyme formulation corresponding to this dosage based on the concentration of nerve agent neutralizing enzyme in a formulation of the invention, In another embodiment of the present invention, additional dosages may be administered if normal physiological functions have not been restored.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing the invention in spirit or in scope. The invention is therefore to be limited only by the terms of the appended claims along with the full scope of equivalents to which those claims are entitled.

### Example I

### Protection from Cutaneous Nerve Agents

This example describes how recombinant human BChE (see PCT Publication No. NO WO 03/054182) can be administered via inhalation to protect individuals from the toxic effects of cutaneous nerve agents exposure.

VX (Q-ethyl-S-(2-iisopropylaminoethyl)methyl phosphonothiolate or ethyl-S-dimethylaminoethyl methylphosphonothiolate) is a very toxic organophosphate nerve agent, which by virtue of its low vapor pressure, is a liquid at room temperature. VX is rapidly absorbed into the bloodstream upon exposure to skin, and subsequently results in perfusion of the central and peripheral nervous systems. VX inactivates acetylcholinesterase and induces a cholinergic crisis.

Exposure to Nerve Agent VX

Subjects are challenged with two LD5O's, e.g., two lethal doses, of VX. The VX challenge may be administered by any method known in the art, preferably by cutaneous exposure. An amount of VX equivalent to two LD5Os may be determined by one skilled in the art, based upon the toxicity of the nerve agent, the method of administration, and the weight of the subject.

Preparation of Cholinesterase

Lyophilized BChE (available from Nexia Bioscieiices under the trade name Protexia®) is prepared with an appropriate excipient to produce particles of approximately 1 pun (range 0.01 to 4 μm) in diameter. Methods of generating particles of a predetermined size are well known in the art, and include but are not limited to, milling, grinding, and/or sieving.

A dose of 150 mg of BChE has been previously shown to provide stoichiometric protection against in monkeys exposed to VX (Raveh et. al., 1997; Toxicol. Appl. Pharmacol. 145:43-53). The known mechanism of action of BChE catabolism of VX is via an esterase reaction which cleaves the VX into 2 inactive chemicals. Often the enzyme is phosphorylated or alkylphosphorylated by one of these reaction products and rendered inactive (Goodman and Oilman, 1980; The Pharmacological Basis of Therapeutics, MacMillan Publishing Co, New York).

Administration of Butyrvlcholinesterase

At a predetermined time following the VX challenge, the powdered formulation of BChE is administered via a pulmonary delivery device, calibrated such that about 2 to about 250 mg of BChE is released into the lungs in one or more inhalations. By way of example, a DPI calibrated to administer 30 mg of BC1XE per inhalation can be utilized to administer 150 mg of BChE in five (5) inhalations. Larger doses may be administered by increasing the number of inhalations, e.g., 210 mg of BChE can be administered in seven (7) inhalations. Dose size may be fine tuned by adjusting the dose of BChE administered per inhalation and adjusting the number of inhalations, e.g., an MDI calibrated to administer 5 mg of BChE can be utilized to deliver 160 mg of BChE in 32 inhalations. Alternatively, BChE may be delivered to an anesthetized subject by means of a nebulizer, intubation, or gavage.

The degree of inhibition by VX can be readily estimated by commercially available blood cholinesterase tests, An in vitro assay for the quantitative determination of cholinesterase in human serum and plasma is available from Roche (Roche CITE Cholinesterase kit Cat, No, 11489259 or 11489445), and can be practiced by those skilled in the art utilizing routine techniques and equipment well known in the art. Blood samples may be taken prior to VX challenge, immediately following VX challenge, prior to administration of BChE, immediately following administration of BChE, and at predetermined intervals following administration of BChE. The level of cholinesterase in the samples may be determined, and compared to a control subject that has not been challenged with VX nor administered BChE. Additional control subjects may be used that are challenged with VX but not administered BChE or have not been challenged with VX but are administered BChE.

The efficacy of BCIIE treatment can also be gauged by measuring the physiological manifestations of VX poisoning, which includes apnea, miosis, pupillary constriction or “pin-point pupils,” salivation, tremors, and central nervous system depression. Measurements in the reduction of severity of these physical manifestations following administration of BChE may be performed. For example, the efficacy of cholinesterase treatment can be measured by monitoring pupillary response of the subject. If normal physiological responses are not achieved, additional doses of BChE may be administered as previously described until normal physiological responses are achieved, Efficacy of the treatment can be determined by comparing the reduction in severity of these physical manifestations following administration of BChE to a control subject that has not been challenged with VX nor administered BChE. Additional control subjects may be used that are challenged with VX but not administered BChE, or have not been challenged with VX but are administered BChE.

Those skilled in the art will recognize that other nerve agent neutralizing enzymes may be utilized instead of BChE. Accordingly, determination of dosage will vary depending on the nerve agent neutralizing enzyme used. In the case of nerve agent neutralizing enzymes that are not inactivated by organophosphorus compounds, the dosage may be significantly smaller. Determination of the appropriate dosage will require no more than routine experimentation for those skilled in the art.

### Example 2

### Protection from Hi2h Vapor Pressure Nerve Agents

This example describes how recombinant human BChE can be administered via inhalation to protect individuals from the toxic effects of nerve agents exposure to the lungs. Sarin is a nerve agent that can be delivered in a gaseous form.

Lyophilized BChE is prepared and administered as described in Example 1. Determination of the dosage of BChE is performed as described in Example 1.

At a predetermined time following administration of BChE, an amount of sarin equivalent to two LD5Os is administered by inhalation to a subject. Determination of two LD5Os can be performed as described in Example I. Administration by inhalation may be performed by any means known in the art, including but not limited to inhalers, masks, intratracheal intubation, or gassing chambers.

The degree of inhibition of sarin gas can be determined as described in Example 1. Symptoms of exposure to sarin gas include runny nose, watery eyes, miosis, eye pain, blurred vision, drooling and excessive sweating, cough, chest tightness, rapid breathing, diarrhea, increased urination, confusion, drowsiness, xveakness, headache, nausea, vomiting, and/or abdominal pain, changes in heart rate, and changes in blood pressure. Measurement of the reduction in severity of sarin gas toxicity can be performed by monitoring the physical manifestations of poisoning, as described in Example I.

BChE administered via inhalation is too large to traverse the lung's epithelial cells, resulting in localization of BChE in the lungs. BChE localized in the lungs will act as a chemical baffler that will react with the sam, and will prevent significant amounts of nerve agents moving into the blood. Sarin neutralization occurs on the lung's epithelial cells, and does not result in the nerve agents moving into the blood. Conversely, localization of the BChE in the lungs does not require movement of the BChE from the blood onto the lung's epithelial cells.

Those skilled in the art will recognize that other nerve agent neutralizing enzymes may be utilized instead of BChE. Accordingly, determination of dosage will vary depending on the nerve agent neutralizing enzyme used. In the case of nerve agent neutralizing enzymes that are not inactivated by organophosphorus compounds, the dosage may be significantly smaller. Determination of the appropriate dosage will require no more than routine experimentation for those skilled in the art.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

What is claimed is:

1. A method of treating nerve agent poisoning in a subject which method comprises providing an effective amount of a nerve agent neutralizing enzyme to pulmonary epithelium of the subject by inhalation.

2. The method of claim 1 wherein the nerve agent neutralizing enzyme is present in a particle of a size of about 0.01 μm to about 4 μm.

3. The method of claim 1 wherein the nerve agent neutralizing enzyme is an aerosol form.

4. The method of claim 1 wherein the nerve agent neutralizing enzyme is in liquid form.

5. The method of claim 1 wherein the nerve agent neutralizing enzyme is in dry powder form.

6. The method of claim 1 wherein the nerve agent neutralizing enzyme further comprises an excipient.

7. The method of claim 1 wherein the nerve agent neutralizing enzyme is administered with a nebulizer

8. The method of claim 1 wherein the nerve agent neutralizing enzyme is administered with an inhaler.

9. The method of claim 1 wherein the subject is a human.

10. The method of claim 1 wherein the nerve agent neutralizing enzyme is selected from the group consisting of cholinesterases, aryldialkylphosphatases, organophosphate hydrolases, carboxylesterases, triesterases, phosphotriesterases, arylesterases, paraoxonases, diisopropylfluorophosphatases, and organophosphate acid anhydrases.

11. The method of claim 10 wherein the nerve agent neutralizing enzyme is butyrylcholinesterase.

12. The method of claim 11 wherein the dose is about 150 mg.

13. The method of claim 10 wherein the nerve agent neutralizing enzyme is acetylcholinesterase.

14. The method of claim 10 wherein the nerve agent neutralizing enzyme is acetylcholinesterase.

15. The method of claim 10 wherein the nerve agent neutralizing enzyme is a carboxylesterase.

16. The method of claim 10 wherein the nerve agent neutralizing enzyme is a aryldialkylphosphatases.

17. The method of claim 10 wherein the nerve agent neutralizing enzyme is a organophosphate hydrolase.

18. The method of claim 10 wherein the nerve agent neutralizing enzyme is a triesterase.

19. The method of claim 10 wherein the nerve agent neutralizing enzyme is a phosphotriesterase.

20. The method of claim 10 wherein the nerve agent neutralizing enzyme is a arylesterases.

21. The method of claim 10 wherein the nerve agent neutralizing enzyme is a paraoxonase.

22. The method of claim 10 wherein the nerve agent neutralizing enzyme is a diisopropylfluorophosphatase

23. The method of claim 10 wherein the nerve agent neutralizing enzyme is a organophosphate acid anhydrase.

24. A pharmaceutical dosage form for delivery of a nerve agent neutralizing enzyme to the pulmonary system of a mammal, the form comprising a therapeutically effective amount of a cholinesterase and a pharmaceutically acceptable carrier or diluent, wherein said dosage form is contained in an inhaler or nebulizer.

25. The pharmaceutical dosage form of claim 24 wherein the nerve agent neutralizing enzyme has a particle size of about 0.01 μm to about 4 μm.

26. The pharmaceutical dosage form of claim 24 wherein said dosage form is an aerosol form.

27. The pharmaceutical dosage form of claim 24 wherein said dosage form is a liquid.

28. The pharmaceutical dosage form of claim 24 wherein said dosage form is a dry powder.

29. The pharmaceutical dosage form of claim 24 wherein said dosage form further comprises an excipient.

30. The pharmaceutical dosage form of claim 24 wherein said mammal is a human.

31. The pharmaceutical dosage form of claim 24, further comprising a carbamate, an anti-muscarinic agent, a cholinesterase reactivators, or an anticonvulsive agent.

32. The pharmaceutical dosage form of claim 24 wherein the nerve agent neutralizing enzyme selected from the group consisting of cholinesterases, aryldialkylphosphatases, organophosphate hydrolases, carboxylesterases, triesterases, phosphotriesterases, arylesterases, paraoxonases, diisopropylfluorophosphatases, and organophosphate acid anhydrases.

33. The pharmaceutical dosage form of claim 32 wherein the nerve agent neutralizing enzyme is butyrylcholinesterase.

34. The pharmaceutical dosage form of claim 32 wherein the nerve agent neutralizing enzyme is acetyleholinesterase.

35. The pharmaceutical dosage form of claim 32 wherein the nerve agent neutralizing enzyme is aryldialkylphosphatases.

36. The pharmaceutical dosage form of claim 32 wherein the nerve agent neutralizing enzyme is a paraoxonase.

37. The pharmaceutical dosage form of claim 32 wherein the nerve agent neutralizing enzyme is a carboxylesterase.

38. The pharmaceutical dosage form of claim 32 wherein the nerve agent neutralizing enzyme is a organophosphate hydrolase (OPH).

39. The pharmaceutical dosage form of claim 32 wherein the nerve agent neutralizing enzyme is a triesterase.

40. The pharmaceutical dosage form of claim 32 wherein the nerve agent neutralizing enzyme is a phosphotriesterase.

41. The pharmaceutical dosage form of claim 32 wherein the nerve agent enzyme is a arylesterases.

42. The pharmaceutical dosage form of claim 32 wherein the nerve agent neutralizing enzyme is a diisopropylfluorophosphatase.

43. The pharmaceutical dosage form of claim 32 wherein the nerve agent neutralizing enzyme is an organophosphate acid anhydrase.

44. A pharmaceutical formulation for delivery of a cholinesterase to the pulmonary system of a mammal comprising cholincsterase particles having a diameter of about 0.01 μm to about 4 μm and a pharmaceutically acceptable diluent or carrier.

45. The pharmaceutical formulation of claim 44 wherein the particles are about 1 μm in diameter.

46. The pharmaceutical formulation of claim 44 wherein said formulation is an aerosol.

47. The pharmaceutical formulation of claim 44 wherein said formulation is a dry powder.

48. The pharmaceutical formulation form of claim 44 wherein the nerve agent neutralizing enzyme is butyrylcholinesterase.

49. The pharmaceutical formulation form of claim 44 wherein the nerve agent neutralizing enzyme is acetylcholinesterase.

50. The pharmaceutical forniulation form of claim 44 wherein the nerve agent neutralizing enzyme is a paraoxonase.

51. The pharmaceutical formulation form of claim 44 wherein the nerve agent neutralizing enzyme is a carboxylesterase.

52. The pharmaceutical formulation form of claim 44 wherein the nerve agent neutralizing enzyme is a organophosphate hydrolases (OPH).

53. The pharmaceutical fommlation form of claim 44 wherein the nerve agent neutralizing enzyme is a triesterases.

54. The pharmaceutical formulation form of claim 44 wherein the nerve agent neutralizing enzyme is a phosphotriesterases

55. The pharmaceutical formulation form of claim 44 wherein the nerve agent neutralizing enzyme is a diisopropyl fluorophosphatase.

56. The pharmaceutical formulation form of claim 44 wherein the nerve agent neutralizing enzyme is a organophosphate acid anhydrases.
